SEQUENCE LISTING (8 sequences)

SEQ ID NO: 1 - DNA, 1425 bp, Acremonium strictum
aactcaatcaacgcctgtctcgccgccgccgacgtcgagttccacgaggaagactccgagggctgggagatggacggcacagccttcaacctccgcgtcgactacgacccggtcgccattgccatccctcgctccaccgaggacatcgctgccgctgttcagtgcggtcttgatgctggtgtgcagatctcggcaaagggtggtggtcacagttatggttcttatgggtttggtggtgaggatggtcatttgatgttggagttggatcgtatgtaccgtgtgtcggttgatgatgataatgtcgcgactattcagggtggtgctcgtcttggatacactgctcttgagcttcttgaccagggtaaccgtgccctgactcacggtacctgccctgctgtcggtatcggtggccacgttctcggtggcggctacgggtttgctacccacacccacggtctgactctcgactggctcgtcggtgccaccgtcgtcctggccgatgcctccatcgtgcacgtctccaagaccgagaacgcagatctcttctgggccctccgcggtggcggcggtggtttcgccatcgtctccgagttcgagttcaacaccttcgaggctccggagatcatcaccacctaccaggtcaccaccacctggaaccggaaacagcacgttgccggtctcaaggctctccaggactgggctgagaacaccatgcccagggagctcagcatgcgtcttgagatcaacgccaatgccctcaattgggagggtaactacttcggtaacgccaaggatttgaagaaggttcttcagcctatcatgaagaaggctggtggcaagtctaccatctctaagcttgttgagaccgattggtatggccagatcaacacctacttgtacggtgctgacctcaacattacctacaactacgacgtccacgagtacttctacgccaacagcttgaccgccccccgcctctccgatgaagccatctccgccttcgtcgactacaaattcgacaactcctccgtccgccccggccgcggctggtggatccaatgggacttccacggcggcaagaactccgccctcgcctcccactccaacgacgagaccgcctacgcccaccgcgaccagctctggctctggcagttctacgacaacatctacgactacgagaacaacacctctccctaccctgagagcgggttcgagttcatgcagggttttgtcgccactattgaagacacgcttcctgaggacaggaagggcaagtacttcaactatgctgatactacgcttgataaggaggaggcgcagaagttgtactggaggggtaaccttgagaagctgcaggctatcaaggccaagtatgaccctgaggatgtgtttggtaatgttgtctctgttgagcccattgcctag

SEQ ID NO: 2 - protein, 474 aa, Acremonium strictum
NSINACLAAADVEFHEEDSEGWEMDGTAFNLRVDYDPVAIAIPRSTEDIAAAVQCGLDAGVQISAKGGGHSYGSYGFGGEDGHLMLELDRMYRVSVDDDNVATIQGGARLGYTALELLDQGNRALTHGTCPAVGIGGHVLGGGYGFATHTHGLTLDWLVGATVVLADASIVHVSKTENADLFWALRGGGGGFAIVSEFEFNTFEAPEIITTYQVTTTWNRKQHVAGLKALQDWAENTMPRELSMRLEINANALNWEGNYFGNAKDLKKVLQPIMKKAGGKSTISKLVETDWYGQINTYLYGADLNITYNYDVHEYFYANSLTAPRLSDEAISAFVDYKFDNSSVRPGRGWWIQWDFHGGKNSALASHSNDETAYAHRDQLWLWQFYDNIYDYENNTSPYPESGFEFMQGFVATIEDTLPEDRKGKYFNYADTTLDKEEAQKLYWRGNLEKLQAIKAKYDPEDVFGNVVSVEPIA

SEQ ID NO: 3 - DNA, 1425 bp, Acremonium strictum
aactcaatcaacgcctgtctcgccgccgccgacgtcgagttccacgaggaagactccgagggctgggagatggacggcacagccttcaacctccgcgtcgactacgacccggtcgccattgccatccctcgctccaccgaggacatcgctgccgctgttcagtgcggtcttgatgctggtgtgcagatctcggcaaagggtggtggtcacagttatggttcttatgggtttggtggtgaggatggtcatttgatgttggagttggatcgtatgtaccgtgtgtcggttgatgatgataatgtcgcgactattcagggtggtgctcgtcttggatacactgctcttgagcttcttgaccagggtaaccgtgccctgactcacggtacctgccctgctgtcggtatcggtggccacgttctcggtggcggctacgggtttgctacccacacccacggtctgactctcgactggctcgtcggtgccaccgtcgtcctggccgatgcctccatcgtgcacgtctccaagaccgagaacgcagatctcttctgggccctccgcggtggcggcggtggtttcgccatcgtctccgagttcgagttcaacaccttcgaggctccggagatcatcaccacctaccaggtcaccaccacctggaaccggaaacagcacgttgccggtctcaaggctctccaggactgggctgagaacaccatgcccagggagctcagcatgcgtcttgagatcaacgccaatgccctcaattgggagggtaactacttcggtaacgccaaggatttgaagaaggttcttcagcctatcatgaagaaggctggtggcaagtctaccatctctaagcttgttgagaccgattggtatggccagatcaacacctacttggccggtgctgacctcaacattacctacaactacgacgtccacgagtacttctacgccaacagcttgaccgccccccgcctctccgatgaagccatctccgccttcgtcgactacaaattcgacaactcctccgtccgccccggccgcggctggtggatccaatgggacttccacggcggcaagaactccgccctcgcctcccactccaacgacgagaccgcctacgcccaccgcgaccagctctggctctggcagttctacgacaacatctacgactacgagaacaacacctctccctaccctgagagcgggttcgagttcatgcagggttttgtcgccactattgaagacacgcttcctgaggacaggaagggcaagtacttcaactatgctgatactacgcttgataaggaggaggcgcagaagttgtactggaggggtaaccttgagaagctgcaggctatcaaggccaagtatgaccctgaggatgtgtttggtaatgttgtctctgttgagcccattgcctag

SEQ ID NO: 4 - DNA, 1425 bp, Acremonium strictum
aactcaatcaacgcctgtctcgccgccgccgacgtcgagttccacgaggaagactccgagggctgggagatggacggcacagccttcaacctccgcgtcgactacgacccggtcgccattgccatccctcgctccaccgaggacatcgctgccgctgttcagtgcggtcttgatgctggtgtgcagatctcggcaaagggtggtggtcacagttatggttcttatgggtttggtggtgaggatggtcatttgatgttggagttggatcgtatgtaccgtgtgtcggttgatgatgataatgtcgcgactattcagggtggtgctcgtcttggatacactgctcttgagcttcttgaccagggtaaccgtgccctgactcacggtacctgccctgctgtcggtatcggtggccacgttctcggtggcggctacgggtttgctacccacacccacggtctgactctcgactggctcgtcggtgccaccgtcgtcctggccgatgcctccatcgtgcacgtctccaagaccgagaacgcagatctcttctgggccctccgcggtggcggcggtggtttcgccatcgtctccgagttcgagttcaacaccttcgaggctccggagatcatcaccacctaccaggtcaccaccacctggaaccggaaacagcacgttgccggtctcaaggctctccaggactgggctgagaacaccatgcccagggagctcagcatgcgtcttgagatcaacgccaatgccctcaattgggagggtaactacttcggtaacgccaaggatttgaagaaggttcttcagcctatcatgaagaaggctggtggcaagtctaccatctctaagcttgttgagaccgattggtatggccagatcaacacctacttgaacggtgctgacctcaacattacctacaactacgacgtccacgagtacttctacgccaacagcttgaccgccccccgcctctccgatgaagccatctccgccttcgtcgactacaaattcgacaactcctccgtccgccccggccgcggctggtggatccaatgggacttccacggcggcaagaactccgccctcgcctcccactccaacgacgagaccgcctacgcccaccgcgaccagctctggctctggcagttctacgacaacatctacgactacgagaacaacacctctccctaccctgagagcgggttcgagttcatgcagggttttgtcgccactattgaagacacgcttcctgaggacaggaagggcaagtacttcaactatgctgatactacgcttgataaggaggaggcgcagaagttgtactggaggggtaaccttgagaagctgcaggctatcaaggccaagtatgaccctgaggatgtgtttggtaatgttgtctctgttgagcccattgcctag

SEQ ID NO: 5 - DNA, 1425 bp, Acremonium strictum
aactcaatcaacgcctgtctcgccgccgccgacgtcgagttccacgaggaagactccgagggctgggagatggacggcacagccttcaacctccgcgtcgactacgacccggtcgccattgccatccctcgctccaccgaggacatcgctgccgctgttcagtgcggtcttgatgctggtgtgcagatctcggcaaagggtggtggtcacagttatggttcttatgggtttggtggtgaggatggtcatttgatgttggagttggatcgtatgtaccgtgtgtcggttgatgatgataatgtcgcgactattcagggtggtgctcgtcttggatacactgctcttgagcttcttgaccagggtaaccgtgccctgactcacggtacctgccctgctgtcggtatcggtggccacgttctcggtggcggctacgggtttgctacccacacccacggtctgactctcgactggctcgtcggtgccaccgtcgtcctggccgatgcctccatcgtgcacgtctccaagaccgagaacgcagatctcttctgggccctccgcggtggcggcggtggtttcgccatcgtctccgagttcgagttcaacaccttcgaggctccggagatcatcaccacctaccaggtcaccaccacctggaaccggaaacagcacgttgccggtctcaaggctctccaggactgggctgagaacaccatgcccagggagctcagcatgcgtcttgagatcaacgccaatgccctcaattgggagggtaactacttcggtaacgccaaggatttgaagaaggttcttcagcctatcatgaagaaggctggtggcaagtctaccatctctaagcttgttgagaccgattggtatggccagatcaacacctacttgtacggtgctgacctcaacattacctacaactacgacgtccacgagtacttctacgccaacagcttgaccgccccccgcctctccgatgaagccatctccgccttcgtcgactacaaattcgacaactcctccgtccgccccggccgcggctggttcatccaatgggacttccacggcggcaagaactccgccctcgcctcccactccaacgacgagaccgcctacgcccaccgcgaccagctctggctctggcagttctacgacaacatctacgactacgagaacaacacctctccctaccctgagagcgggttcgagttcatgcagggttttgtcgccactattgaagacacgcttcctgaggacaggaagggcaagtacttcaactatgctgatactacgcttgataaggaggaggcgcagaagttgtactggaggggtaaccttgagaagctgcaggctatcaaggccaagtatgaccctgaggatgtgtttggtaatgttgtctctgttgagcccattgcctag

SEQ ID NO: 6 - protein, 474 aa, Acremonium strictum
NSINACLAAADVEFHEEDSEGWEMDGTAFNLRVDYDPVAIAIPRSTEDIAAAVQCGLDAGVQISAKGGGHSYGSYGFGGEDGHLMLELDRMYRVSVDDDNVATIQGGARLGYTALELLDQGNRALTHGTCPAVGIGGHVLGGGYGFATHTHGLTLDWLVGATVVLADASIVHVSKTENADLFWALRGGGGGFAIVSEFEFNTFEAPEIITTYQVTTTWNRKQHVAGLKALQDWAENTMPRELSMRLEINANALNWEGNYFGNAKDLKKVLQPIMKKAGGKSTISKLVETDWYGQINTYLAGADLNITYNYDVHEYFYANSLTAPRLSDEAISAFVDYKFDNSSVRPGRGWWIQWDFHGGKNSALASHSNDETAYAHRDQLWLWQFYDNIYDYENNTSPYPESGFEFMQGFVATIEDTLPEDRKGKYFNYADTTLDKEEAQKLYWRGNLEKLQAIKAKYDPEDVFGNVVSVEPIA

SEQ ID NO: 7 - protein, 474 aa, Acremonium strictum
NSINACLAAADVEFHEEDSEGWEMDGTAFNLRVDYDPVAIAIPRSTEDIAAAVQCGLDAGVQISAKGGGHSYGSYGFGGEDGHLMLELDRMYRVSVDDDNVATIQGGARLGYTALELLDQGNRALTHGTCPAVGIGGHVLGGGYGFATHTHGLTLDWLVGATVVLADASIVHVSKTENADLFWALRGGGGGFAIVSEFEFNTFEAPEIITTYQVTTTWNRKQHVAGLKALQDWAENTMPRELSMRLEINANALNWEGNYFGNAKDLKKVLQPIMKKAGGKSTISKLVETDWYGQINTYLNGADLNITYNYDVHEYFYANSLTAPRLSDEAISAFVDYKFDNSSVRPGRGWWIQWDFHGGKNSALASHSNDETAYAHRDQLWLWQFYDNIYDYENNTSPYPESGFEFMQGFVATIEDTLPEDRKGKYFNYADTTLDKEEAQKLYWRGNLEKLQAIKAKYDPEDVFGNVVSVEPIA

SEQ ID NO: 8 - protein, 474 aa, Acremonium strictum
NSINACLAAADVEFHEEDSEGWEMDGTAFNLRVDYDPVAIAIPRSTEDIAAAVQCGLDAGVQISAKGGGHSYGSYGFGGEDGHLMLELDRMYRVSVDDDNVATIQGGARLGYTALELLDQGNRALTHGTCPAVGIGGHVLGGGYGFATHTHGLTLDWLVGATVVLADASIVHVSKTENADLFWALRGGGGGFAIVSEFEFNTFEAPEIITTYQVTTTWNRKQHVAGLKALQDWAENTMPRELSMRLEINANALNWEGNYFGNAKDLKKVLQPIMKKAGGKSTISKLVETDWYGQINTYLYGADLNITYNYDVHEYFYANSLTAPRLSDEAISAFVDYKFDNSSVRPGRGWFIQWDFHGGKNSALASHSNDETAYAHRDQLWLWQFYDNIYDYENNTSPYPESGFEFMQGFVATIEDTLPEDRKGKYFNYADTTLDKEEAQKLYWRGNLEKLQAIKAKYDPEDVFGNVVSVEPIA

(19) United States
(12) Patent Application Publication
(10) Pub. No.: US 2014/0057332 A1
(43) Pub. Date: Feb. 27, 2014

(54) GLUCO-OLIGOSACCHARIDE OXIDASES FROM ACREMONIUM STRICTUM AND USES THEREOF

(71) Applicants: Maryam Foumani, Richmond Hill (CA); Thu V. Vuong, Toronto (CA); Emma R. Master, Toronto (CA)

(72) Inventors: Maryam Foumani, Richmond Hill (CA); Thu V. Vuong, Toronto (CA); Emma R. Master, Toronto (CA)

(73) Assignee: The Governing Council of the University of Toronto, Toronto (CA)

(21) Appl. No.: 14/002,002

(22) PCT Filed: Feb. 28, 2012
(86) PCT No.: PCT/CA2012/000171

§ 371 Date: Nov. 13, 2013

Related U.S. Application Data
(60) Provisional application No. 61/447,550, filed on Feb. 28, 2011.

(51) Int. Cl. C12N 9/04

(52) U.S. Cl. CPC C12N 9/0006
USPC 435/190; 536/23.2

(57) ABSTRACT

This invention provides a description of novel polypeptides and nucleotide sequences having gluco-oligosaccharide oxidase (GOOX) activity. The polypeptides of the invention can be used for enzymatic processes that modify carbohydrates from wood fiber. These polypeptides can be used in the oxidation of C⁶ and C⁵ mono- and oligomeric sugars. These polypeptides can also be used for the oxidation of glucose, xylose, galactose, NAG, xylo-oligosaccharides, cello-oligosaccharides. The novel polypeptides of the invention can be used in a variety of pharmaceutical, agricultural and industrial contexts.

### FIELD OF THE INVENTION

This invention relates to the development of specific gluco-oligosaccharide oxidase (GOOX) variants from an Acremonlum strictum strain, the substrate specificity of the variants, the improvement of GOOX substrate specificity through site-directed mutagenesis, and uses of these novel GOOX variants.

### BACKGROUND OF THE INVENTION

Oxidation of oligo- and poly-saccharides can alter the rheology of corresponding polymers, and be performed as an initial step to subsequent etherification, esterification or amination of hydroxyl groups. TEMPO (2,2,6,6-tetramethylpiperidine-1-oxyl) is a common oxidizing reagent used to convert primary hydroxyl groups of polysaccharides to carboxylic acids. However, TEMPO can compromise the polymerization and/or crystallinity of the starting material, which is problematic when derivatizing oligosaccharide and nanocrystailine substrates. Enzymatic oxidation would enable regioselective modification of highly functionalized carbohydrates without arduous protection/deprotection steps. Mild reaction requirements also mean that loss in the degree of polymerization and crystallinity of oligo- and poly-saccharide substrates can be minimized.

Carbohydrate oxidases (EC 1.1.3) can catalyze the oxidation of the primary hydroxyl (C⁶ in pyranoses), secondary hydroxyls (C², C³ or C⁴) or anomeric carbon hydroxyl (C¹) to an aldehyde, ketone or a lactone (then carboxylic acid), respectively, with concomitant reduction of molecular oxygen to hydrogen peroxide (19). Given the ease of detecting hydrogen peroxide, glucose oxidase (GOX) and pyranose oxidase (PDX) have been widely applied in clinical biosensors. GOX and PDX oxidize the hydroxyl group at the C1 and C2 positions of sugar substrates, respectively, and crystal structures of these enzymes reveal a size exclusion mechanism for substrate binding (7, 23). As a result, the application of GOX and PDX is likely limited to the oxidation of mono- and di-saccharides. By contrast, galactose oxidase (GaOX) oxidizes the hydroxyl group at the C⁶ position of galactose (20) and extensive analyses of GaOX have revealed a comparatively shallow active site, explaining the activity of this enzyme on galactopyranosyl units of galactoglucomannans, in addition to monosaccharides (D/L-galactose) and oligosaccharides with terminal galactopyranosyl units (6).

The activity of GaOX on plant-derived polysaccharides has been demonstrated and used to alter the rheology of polysaccharides containing terminal galactose units (e.g. galactoglucomannan, galactomannan, and xylogiucans) (18). By contrast, oligosaccharide oxidases that oxidize C¹ hydroxyl groups of β-1,4-linked sugars are potentially valuable enzymes for derivatization of xylan and cellulosic substrates. Examples of oligosaccharide oxidases include a cello- and malto-oligosaccharide oxidase from Microdochlum nivale (MnCO) (23), a cello-oligosaccharide oxidase from Paraconlothyrium sp. (PCOX) (12), a chito-oligosaccharide oxidase from Fusarium graminearum (ChitO) (8), and a gluco-oligosaccharide oxidase from Acremonium strictum (GOOX) (15). The protein sequences of MnCO, ChitO and GOOX similarly predict a flavin adenine dinucleotide (FAD)-binding domain and a substrate-binding pocket. Like other flavin carbohydrate oxidases that target the anomeric carbon hydroxyl (C¹), oligosaccharide oxidases are thought to mediate oxldoreductase activity through two half-reactions: 1) oxidation of the reducing sugar to the corresponding lactone, then 2) spontaneous hydrolysis of the lactone product to the corresponding acid (20).

Huang et al. (2005) resolved the crystal structure of GOOX from A. strictum strain T1 and proposed that Tyr429 initiates sugar oxidation by proton abstraction from the C¹ hydroxyl, followed by H¹ hydride transfer to the N⁵ position of the FAD cofactor (10). Notably, the FAD is covalently bound by two amino acids, His70 and Cys130; this unique configuration is predicted to modulate the oxidative potential of the FAD cofactor (11, 13). The crystal structure of GOOX further reveals that similar to cellobiose dehydrogenase and GaOX (6, 7); the enzyme possesses an open carbohydrate-binding groove, allowing the accommodation of oligosaccharide substrates (FIG. 3).

A screening of more than 50 carbohydrates and derivatives show that GOOX oxidizes both α-linked and β-linked glucose substrates, including lactose, malto-oligosaccharides and cello-oligosaccharides (5, 8, 9). The catalytic efficiency of native GOOX purified from A. strictum T1 is highest with cellotriose (13); however, this GOOX did not oxidize xylose, galactose, or many other sugars (15). The impact of temperature and pH on GOOX activity was studied extensively using cello- and maltooligosaccharides (5). In their study, Fan et al. (2000) revealed that the oxidation of maltose was highest between pH 9 to 10.5, but the Kₘ of this reaction was also highest at pH 9. Fan et al. (2000) also demonstrated that the activation energy of GOOX is similar at pH 7 and pH 10, suggesting that the catalytic mechanism of GOOX is retained within this pH range.

While the catalytic mechanism of GOOX has been characterized, residues that affect the substrate preference of this enzyme are still unknown. Given the limited arsenal of biocatalysts that can be applied for oxidative modification of plant-derived oligosaccharides, GOOX variants with gained activity on xylose, galactose, and/or mannose containing substrates would constitute a valuable set of new industrial enzymes.

### SUMMARY OF THE INVENTION

The inventors have demonstrated the purification and substrate specificity of a GOOX variant from an A. strictum strain, and the improvement of its substrate specificity through site-directed mutagenesis.

The recombinant protein of the present invention, hereinafter GOOX-VN, contains fifteen amino acid substitutions compared with the previously reported A. strictum GOOX. These two enzymes share 97% sequence identity; however, only GOOX-VN oxidizes xylose, galactose, and N-acetylglucosamine. Besides monosaccharides, GOOX-VN oxidized xylo-oligosaccharides, including xylobiose and xylotriose with similar catalytic efficiency as for cello-oligosaccharides.

In accordance with another aspect of the present invention, three purified mutant enzymes created in GOOX-VN, identified as Y300A, Y300N and W351F, are provided. Of the three mutant enzymes that were created in GOOX-VN to improve substrate specificity, Y300A and Y300N doubled k_{cat} values for monosaccharide and oligosaccharide substrates.

With this novel substrate specificity, GOOX-VN and its variants are particularly valuable for oxidative modification of cello- and xylo-oligosaccharides.

### BRIEF DESCRIPTION OF THE DRAWINGS

FIG. 1: DNA sequence of GOOX-VN (SEQ ID NO. 1)

FIG. 2: Protein sequence of GOOX-VN (SEQ ID NO. 2)

FIG. 3: Structural model of GOOX-VN (built by the Swiss-Model Workspace using the X-ray structure of GOOX-T1 (PDB ID: 2AXR))

FIG. 4: DNA sequence of Y300A (variant 1) (SEQ ID NO. 3)

FIG. 5: DNA sequence of Y300N (variant 2) (SEQ ID NO. 4)

FIG. 6: DNA sequence of W351F (variant 3) (SEQ ID NO. 5)

FIG. 7: Protein sequence of the three variants of GOOX-VN, including Y300A (A), Y300N (B) and W351F (C) (SEQ ID NOs. 6, 7 and 8)

FIG. 8: Structural model of GOOX-VN showing the location of Y300, W351, and N388 in relation to the intermediate analogue 5-amino-5deoxy-cellobiono-1,5-lactam (ABL) and the FAD cofactor (Hydrogen bonds are shown as dashed lines).

FIG. 9: Docking of monosaccharides to GOOX-VN. Docking positions of glucose (A), xylose (B) and galactose (C); and the side chains of Y300 and W351 were shown. The O⁴ atom of galactose (circled) pointed to the benzene ring of W351, and their distance was 3.1 Å.

FIG. 10: Multiple sequence alignment of GOOX-VN homologues. The alignment between MnCO (CAI94231-2) from Microdochium nivale, ChitO (XP_{—}391174) from Fusarium graminearum, and GOOX-VN was generated using T-coffee. Amino acids, which were mutated, are highlighted with asterisks.

FIG. 11: Residual activity of GOOX-VN (circle), W351F (square), Y300A (cross) and Y300N (triangle) enzymes on 10 mM maltose after incubation at 37° C. in triplicate for up to 1 h.

FIG. 12: SDS-PAGE of purified GOOX-VN and its mutant enzymes. SDS-PAGE was performed using a 12% polyacrylamide gel and proteins were stained with Coomasie Blue. Lane 1: PageRuler™ Plus prestained protein ladder (Fermentas), Lane 2: GOOX-VN enzyme, Lane 3: W351F mutant enzyme, Lane 4: Y300A mutant enzyme, and Lane 5: Y300N mutant enzyme. 0.8 μg of purified protein was applied.

FIG. 13: The formation of derivatized product (m/z 512) in reactions containing GOOX-VN.

FIG. 14: The formation of a new product with mass to charge ratio (m/z) of 699 in reactions containing GOOX-VN.

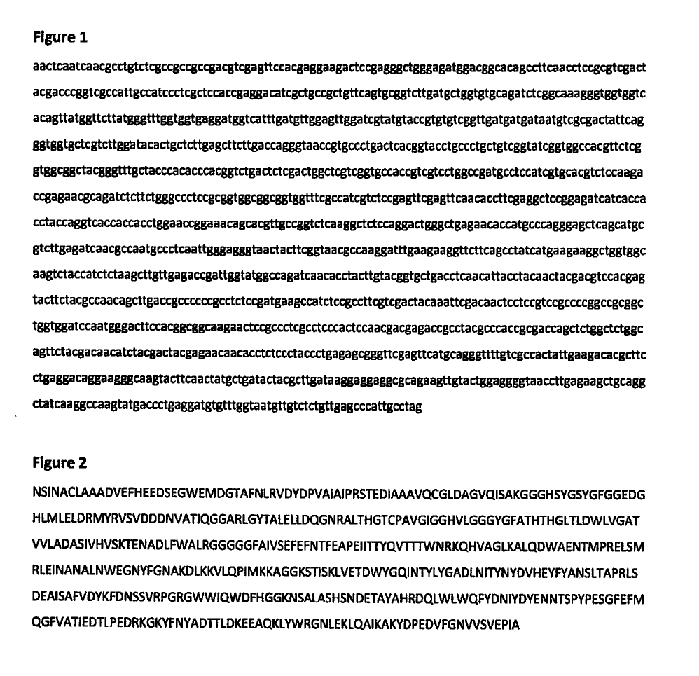

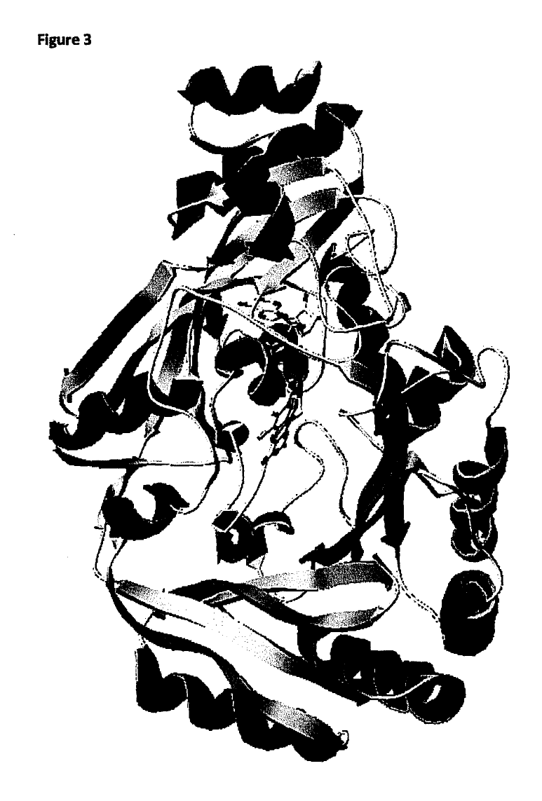

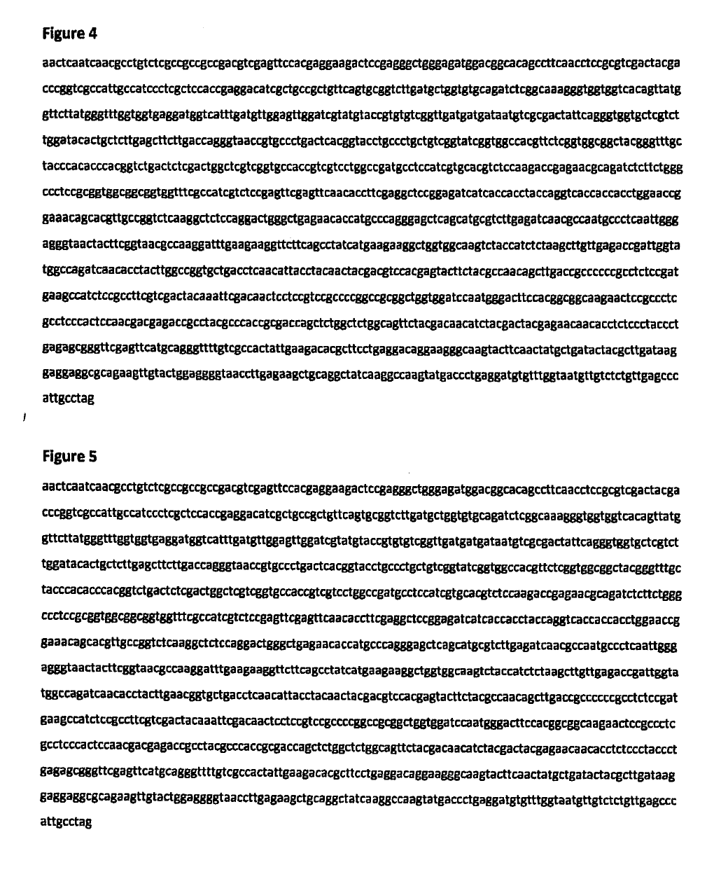

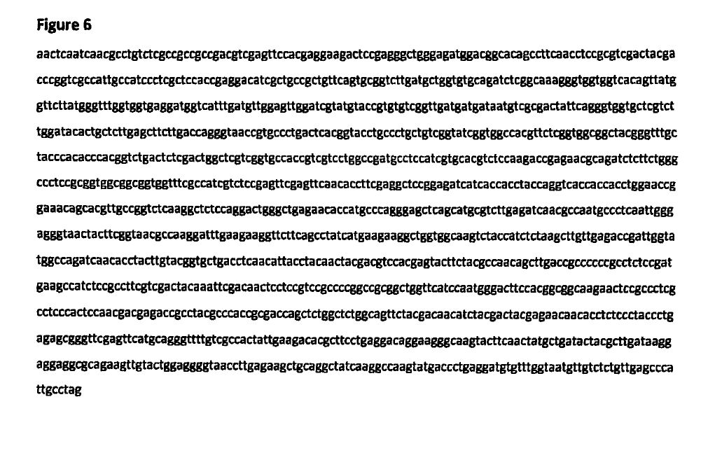

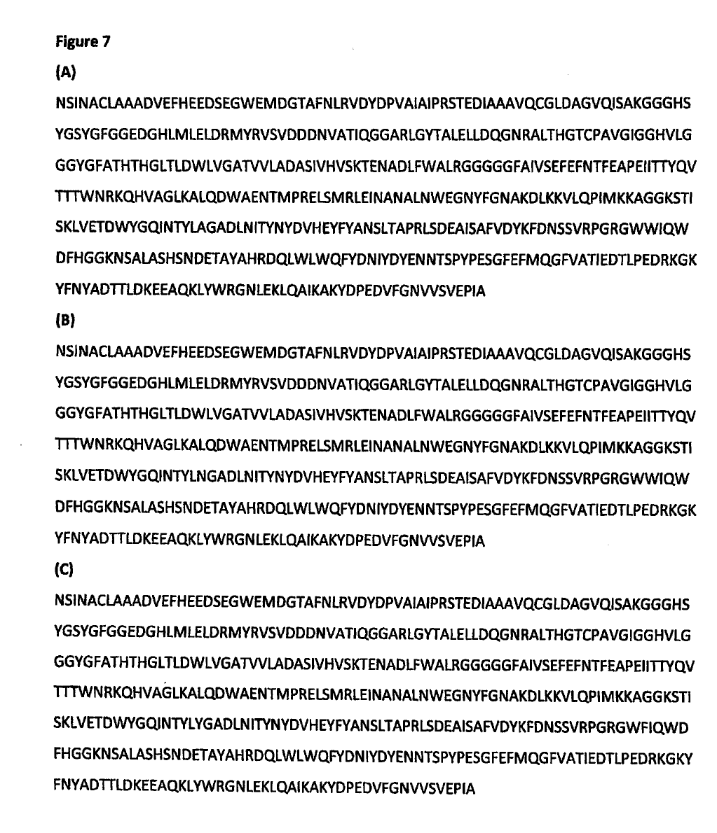

### DETAILED DESCRIPTION

The inventors of the present application have demonstrated that GOOX with different substrate specificity were produced by different strains of A. strictum, widening the application of GOOX from A. strictum for the oxidation of mono- and oligo-saccharides. In addition to glucose, maltose and cello-oligosaccharides, the new GOOX-VN oxidized xylo-oligosaccharides, galactose, and N-acetylglucosamine. This was not detected in GOOX from previous studies. Y300A and Y300N substitutions increased the catalytic activity of GOOX-VN on all substrates, and gained low activity on mannose. Rational engineering approaches are now being applied to decrease the Kₘ of GOOX-VN and its mutant enzymes on oligomeric substrates. In particular, given the consistency between computational docking analyses and experimental data reported in the current study, docking analyses will be used to predict the effect of selected amino acid substitutions on the binding affinity, conformation, and orientation of substrates bound by GOOX-VN and variant enzymes. It is anticipated that resulting carbohydrate oxidases will constitute new tools for the quantitative detection and derivatization of carbohydrates.

Variations of GOOX.

The GOOX gene cloned from A. strictum type strain CBS 346.70 encoded a mature protein containing 474 amino acids, which is the same length as a previously reported GOOX isolated from A. strictum strain T1 (hereafter GOOX-T1) (13,15). However, there were 15 amino acid substitutions between the two proteins, 13 resulting from differences in corresponding wild-type gene sequences, and 2 (A38V and S388N) resulting from random mutations introduced during the construction of the expression system (Table 6). The new GOOX with V38 and N388, hereafter GOOX-VN, shares 97% sequence identity with the reported GOOX-T1 (13), and it has a similar fold to GOOX-T1.

Production of Recombinant Protein.

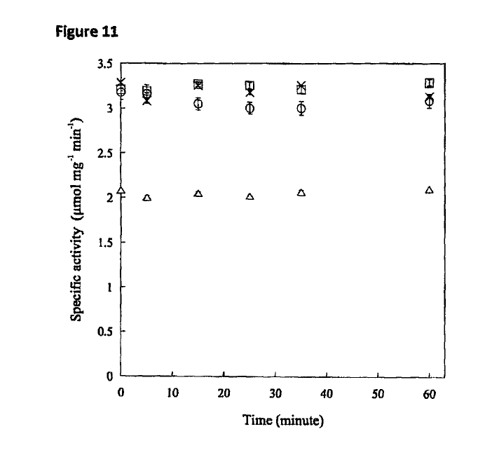

The recombinant expression of GOOX-VN in P. pastoris GS115 was highest after three days of incubation with 0.5% methanol. Proteins were purified to more than 95% homogeneity by affinity chromatography; similar to previous reports of recombinant GOOX-T1 expression by P. pastoris (11). Approximately 1.5 mg L⁻¹ of purified GOOX-VN was recovered, and after confirming that one freeze-thaw cycle did not affect enzyme activity, the purified enzyme was stored as 20 μL aliquots (˜4 μg) at −80° C. The enzyme remained active following pre-incubation at 37° C. for 60 min (FIG. 11).

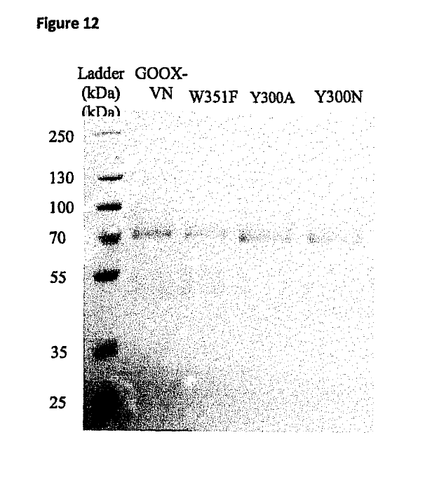

The deduced molecular mass of the mature protein with a 0-mg epitope and a polyhistidine tag is approximately 56 kDa (Protean, DNASTAR-Lasergene), which is less than the electrophoretic molecular weight of purified GOOX-VN (˜70 kDa) (FIG. 12). By comparison, the reported molecular weight of GOOX-T1 determined by size exclusion chromatography is approximately 61 kDa (13). Recombinant proteins expressed in P. pastoris G5115 can be N-glycosylated with high-mannose-type structures containing 8 to 14 Man residues (2, 9). And NetNGlyc predicted three N-glycosylation sites in GOOX-VN, including N305, N341, and N394, which are all located in exposed loop regions. Still, the molecular weight of deglycosylated GOOX-VN was ˜60 kDa, suggesting that other post-translational modifications, including O-glycosylation and/or phosphorylation, probably occurred (3, 4, 14). Notably, deglycosylation of GOOX-VN under native conditions did not cause a detectable loss in enzyme activity (Table 7).

Novel Substrate Specificity.

GOOX-VN oxidase activity was evaluated using glucose, xylose, galactose, N-acetylglucosamine (NAG), mannose, and arabinose. Glucose, xylose, galactose, and NAG were oxidized by the recombinant GOOX-VN, and the highest catalytic efficiency was observed using glucose (Table 1). Previous analyses of GOOX-T1 did not detect activity on xylose, galactose or NAG, and activity was limited to glucose and oligosaccharides with reducing end-glucosyl residues (5, 15). To check whether GOOX-VN can oxidize oligomers of C⁵ sugars, the enzyme was then tested for oxidation of xylo-oligosaccharides. GOOX-VN oxidized xylo-oligosaccharides as efficiently as cello-oligosaccharides (Table 1), and the catalytic efficiency of GOOX-VN on these oligosaccharides was over two orders of magnitude higher than that of the corresponding monomers. These findings show that GOOX-VN has broader substrate specificity than GOOX-T1, and GOOX-VN oxidizes C⁶ and C⁵ mono- and oligomeric sugars.

The broader substrate range of GOOX-VN detected in the current study compared to previous reports using GOOX-T1 is unlikely the result of different assay conditions. While reactions for kinetic analyses of GOOX-VN proceeded for up to 15 min and included substrate concentrations over 500 mM, oxidation of xylose, galactose and NAG by GOOX-VN was detected after 3 min using 10 mM of each sugar, which were the reaction conditions previously used to screen GOOX-T1 activity (13). Furthermore, the k_{cat} value of the recombinant GOOX-T1 on maltose is similar to that of GOOX-VN (361 min⁻¹ and 360.0 min⁻¹, respectively) (13), and GOOX-T1 oxidation of maltose was used by both Lin et al. (15) and Lee et al. (13) to calculate the relative activity of GOOX-T1 on other sugars.

Alternatively, novel substrate specificity of GOOX-VN is likely due to amino acid substitutions in this enzyme. Most substitutions are located on the protein surface or far from the oxidation site (Table 6); however, N388 is positioned on the same β16-sheet as conserved residues Q384 and Y386, which are predicted to participate in substrate binding (11). The side chain of N388 is located near the predicted −2 subsite, within 6.2 Å from the substrate. When comparing the X-ray structures of precursor and mature galactose oxidase from Fusarium spp., Firbank et al. (6) showed that the C_{α} of Tyr290 moved by 6.3 Å and the loop containing this residue could shift up to 8 Å (6). While general loop movement was not observed when comparing GOOX-T1 structures before and after inhibitor binding, the side chain of 5388 in GOOX-T1 turned significantly upon substrate binding to form a weak H-bond with the G349 backbone of the β15-sheet (11). Accordingly, the beneficial effect of the S388N substitution on GOOX-VN activity might be due to the potential of Asn to stabilize substrates that contain fewer hydroxyl groups and/or to stabilize the β16-sheet for substrate binding.

Docking analysis determined that the computational K_{d} for xylose was two times higher than that for glucose, suggesting that low activity on xylose, which does not possess an exocyclic CH₂OH, might be due to weak binding of this substrate by GOOX-VN (Table 8). The Kₘ values for di- and tri-saccharides obtained experimentally, as well as the corresponding K_{d} values derived from the docking models, are an order of magnitude lower than the Kₘ and K_{d} values for monosaccharides (Tables 1, 8). These results support the presence of two glycosyl-binding subsites in the carbohydrate-binding groove of GOOX-VN, which was also predicted by the X-ray structure of GOOX-T1 (11).

Improvement of Substrate Specificity.

The catalytic activity of GOOX-VN on monosaccharides and oligosaccharides was further improved through site-directed mutagenesis. Amino acids targeted for this analysis were chosen by: 1) referencing the published structure of GOOX-T1 (11), and 2) identifying amino acids in GOOX-VN that participate in substrate-binding, which consistently differ from corresponding residues in ChitO from F. graminearum and MnCO from M. nivale.

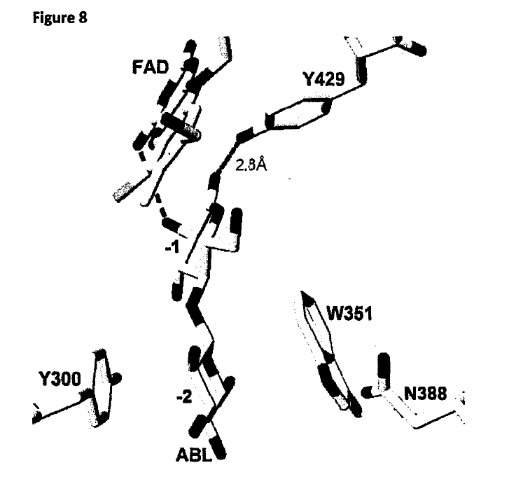

Y300 and W351 are located at the −2 glucosyl-binding subsite (FIG. 8), and likely stabilize oligosaccharide binding through stacking interactions. Y300 is substituted by alanine in ChitO and asparagine in MnCO while W351 is substituted by phenylalanine in MnCO. Since MnCO is distinguished by its activity on galactose, xylose and to some extent on mannose (23), altering the polarity and/or size of Y300 and W351 could increase the activity of GOOX on sugars with an axial OH⁴ group or that lack an exocyclic CH₂OH group. Accordingly, Y300N, Y300A and W351F substitutions were generated in GOOX-VN, and 3 mg L⁻¹, 4 mg L⁻¹ and 1.3 mg L⁻¹ of each purified protein was recovered, respectively. The mutant enzymes remained active after a one hour-pre-incubation at 37° C. (FIG. 11).

The catalytic activity (k_{cat}) of Y300A (Table 2) and Y300N (Table 3) mutant enzymes on all tested monosaccharides and oligosaccharides was approximately two times higher than that of GOOX-VN (Table 1). These two mutant enzymes also gained low activity on mannose. However, the loss in hydrophobic interactions at the −2 subsite also increased the Kₘ and K_{d} values for oligosaccharides, reducing overall catalytic efficiency. These results suggest that Y300 affects substrate positioning relative to the catalytic Y429 residue and the FAD cofactor, and that Y300 contributes to stacking interactions with substrates containing more than two units.

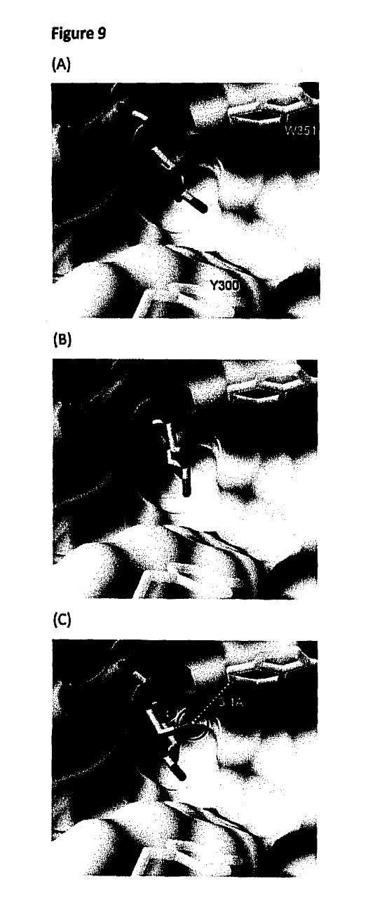

The W351F mutation slightly reduced the catalytic activity of GOOX-VN on all substrates. Like Y300A and Y300N mutations, the W351F mutation also increased the Kₘ values of GOOX-VN with oligomeric substrates (Table 4). These results are consistent with both Y300 and W351 participating in stabilizing stacking interactions with penultimate reducing sugars of oligomeric substrates, which also explains why the impact of these mutations on Kₘ is similar with di- and tri-saccharides (Table 4). Notably, the W351F mutation also increased the Kₘ values of GOOX-VN with glucose and xylose, but decreased the Kₘ of GOOX-VN with galactose, resulting in higher catalytic efficiency with this substrate (Table 4). Docking studies showed that while glucose and xylose binding at the active-site was not restricted, the axial OH⁴ group of galactose points directly towards the benzene ring of tryptophan (FIG. 9), suggesting that the indole structure hinders GOOX-VN binding of sugars with axial OH⁴ groups.

### Example 1

### Cloning of the GOOX-Encoding Gene.

Acremonium strictum type strain CBS 346.70 was obtained from the American Type Culture Collection (ATCC) No.34717. A. strictum was grown on 1 g mL⁻¹ food grade wheat bran at 27° C. for 5 days, harvested by filtration through Miracloth (Calbiochem), and then flash-frozen using liquid nitrogen. Total RNA was extracted from the ground sample using the RNeasy Plant Mini Kit (Qiagen). The full-length cDNA encoding the GOOX protein was isolated using the Long Range 2Step RT-PCR Kit (Qiagen), Briefly, reverse transcription at 42° C. for 90 min was followed by PCR using Pfu DNA polymerase (Agilent Technologies), gene-specific primers (13), and 35 cycles of 93° C. for 30 s, annealing at 56° C. for 40 s, and extension at 72° C. for 2 min. The PCR product was purified using the QIAquick PCR Purification Kit (Qiagen), and then sequenced at the Centre of Applied Genomics (TCAG, the Hospital for Sick Children). The GOOX encoding gene was cloned into the pPICZαA expression vector (Invitrogen), using EcoRI and XbaI and T4 DNA ligase (Invitrogen).

Site-Directed Mutagenesis.

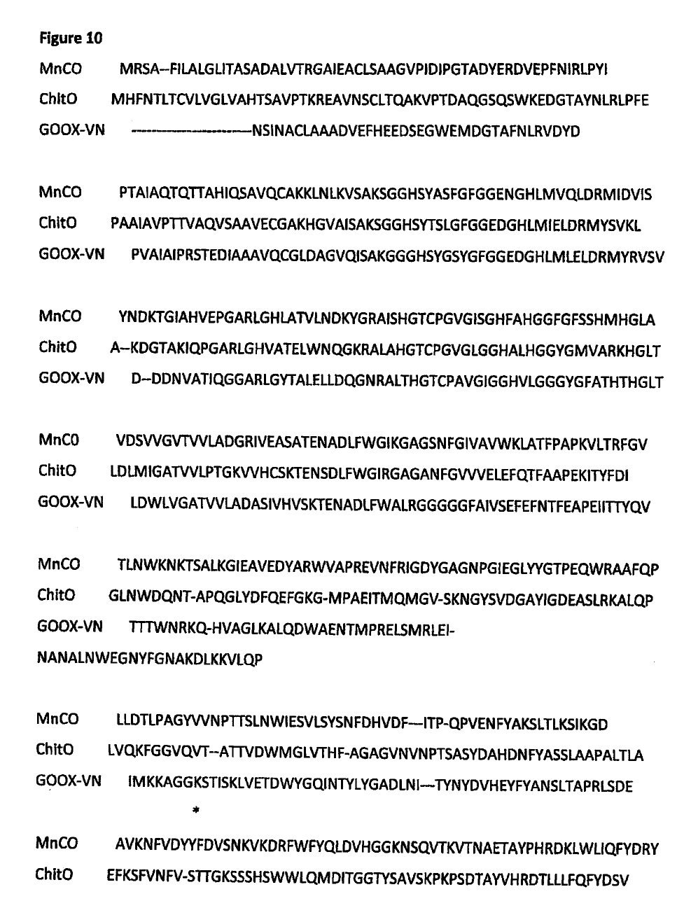

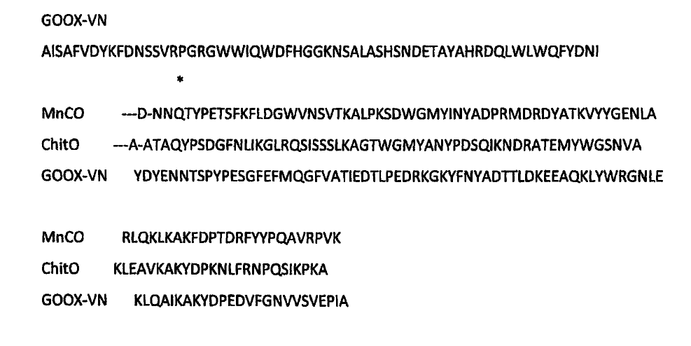

Chito-oligosaccharide oxidase, ChitO, (accession no.: XP_{—}391174) from Fusarium graminearum and a carbohydrate oxidase from Microdochium nivale, MnCO, (accession no.: CAI94231-2) were aligned to GOOX (accession no.: ADI58761) using the Megalign program (DNASTAR-Lasergene) (FIG. 10). Amino acids that were predicted to participate in substrate binding, and that varied between the enzymes analyzed, were selected for site-directed mutagenesis. Mutations Y300A, Y300N and W351F were introduced using mutagenic primers (Table 5). PCR was performed for 14 cycles of 95° C. for 30 s; 55° C. for 1 min; and 68° C. for 5 min, using the QuikChange method (Agilent Technologies). The mutations were confirmed by sequencing (TCAG, the Hospital for Sick Children).

Recombinant Protein Expression.

Mutated plasmids were transformed Into Pichia pastoris GS115 according to the manufacturer's instructions (Invitrogen, Pichia Expression version G). Transformants were selected on buffered minimal methanol medium containing histidine (BMMH, 100 mM potassium phosphate, pH 6.0; 1.34% yeast nitrogen base without amino acids (YNB); 4×10⁵% biotin; 0.5% methanol, 0.004% histidine), and then screened for protein expression by immuno-colony blot using nitrocellulose membranes (0.45 μm, Bio-Rad), anti-Myc antibodies (Sigma), alkaline phosphatase-linked anti-Rabbit IgG conjugates (Sigma), and 5′ bromo-4-chloro-3-indolyl phosphate nitroblue tetrazolium solution (BCIP/NBT, Sigma). Positive transformants were grown overnight in 100 mL of buffered minimal glycerol medium containing histidine (BMGH, 100 mM potassium phosphate, pH 6.0; 1.34% YNB; 4×10⁵% biotin; 1% glycerol, 0.004% histidine) at 30° C. with continuous shaking at 300 rpm. The cells were harvested by centrifugation at 1,500×g for 10 min and suspended in 300 mL of BMMH medium in 1 L-flasks to OD600˜1. Cultures were grown at 30° C. and 300 rpm for 3 days and 0.5% methanol was added every 24 h to induce recombinant protein expression. Levels of recombinant protein expression were monitored every 24 h by activity and SOS-PAGE.

Enzyme Purification.

Supernatants from methanol-induced cultures of P. pastoris expressing recombinant proteins were harvested by centrifugation at 6,000×g for 10 min and filtration through 0.22 μm Sterivex filter units (Millipore). Cleared supernatants were concentrated approximately 150 times using Centricon concentration units (Millipore). Each recombinant protein was purified using a new Ni-NTA resin (Qiagen). Fractions were eluted with 250 mM imidazole and the buffer was replaced by 40 mM Tris-HCl (pH 8.0) using Vivaspin6 concentration units (GE Healthcare). Protein concentration measurements were performed using the Pierce BCA assay (Thermo Scientific) and enzyme purity was verified by SDS-PAGE. In-gel trypsin digestion with sequencing-grade trypsin (Promega), followed by tandem mass spectrometry was performed to confirm the identity of each protein sample. Tryptic fragments were analyzed using the Applied Biosystems/MDS Sciex API QSTAR XL Pulsar System coupled with an Agilent nano HPLC (1100 series) (The Advanced Protein Technology Centre, the Hospital for Sick Children). Proteomic data were analyzed using Scaffold Viewer (www.proteomesoftware.com).

Enzymatic Assays and Kinetic Analyses.

A chromogenic assay was used to measure hydrogen peroxide production (15). Reactions contained 0.1 mM 4aminoantipyrine (4AA), 1 mM phenol, 0.5 U horseradish peroxidase, 40 mM Tris-HCl (pH 8.0), and different substrates were initiated by adding 0.2 μg of enzymes to the 250 μL reaction mixture. The production of H2O2 was coupled to the oxidation of 4aminoantipyrine by horseradish peroxidase and detected at 500 nm. Reactions were incubated at 37° C. for 15 min to measure the specific activity of GOOX on 10 mM of monosaccharide or 1 mM of oligosaccharide. Cello-oligosaccharides were purchased from Sigma (Canada) while xylo-oligosaccharides and manna-oligosaccharides were from Megazyme.

Kinetic parameters were determined with a wide range of substrate concentrations: 0.1 mM to 300 mM glucose, 1 mM to 1500 mM xylose, 1 mM to 600 mM galactose, 1 mM to 600 mM N-acetyl-glucosamine (NAG), 0.1 mM to 300 mM maltose, 5 μM to 1.5 mM cellobiose, 10 μM to 3.5 mM cellotriose, 20 μM to 40 mM xylobiose, and 20 μM to 50 mM xylotriose. At least 12 substrate concentrations were included to obtain kinetic parameters for each substrate. Initial rates were obtained by measuring reaction products every 30 s for 15 min at 37° C. and pH 8.0, and kinetic parameters were calculated using the Michaelis-Menten equation (GraphPad Prism5 Software).

The enzyme stability was evaluated in triplicate by incubating 0.6 μg of each enzyme preparation in 40 mM Tris-HCl buffer (pH 8.0) for 0, 5, 15, 25, 35, and 60 min at 37° C. Residual enzyme activity was measured at 37° C. for 15 min at pH 8.0 using 10 mM maltose and 0.2 μg of protein.

Deglycosylation.

Approximately 2 μg of purified enzyme was treated with PNGaseF (New England Biolabs) using denaturing and native conditions. Samples that were deglycosylated using denaturing conditions were analysed by SDS PAGE, while samples deglycosylated using native conditions were used to evaluate the impact of glycosylation on enzyme activity. The activity of enzymes was measured on 10 mM maltose at 37° C. for 15 min. (Table 7). N-glycosylation was predicted by NetNGlyc (http://www.cbs.dtu.dk/services/NetNGlyc/) while O-glycosylation was predicted by OGPET (http://ogpet.utep.edu/OGPET/).

Substrate Docking.

The structural model of GOOX from A. strictum type strain CBS 346.70 expressed in Pichia was built based on the X-ray structure of A. strictum strain T1 (PDB ID: 2AXR) using the Swiss-Model Workspace (1). The structures of glucose, cellobiose, cellotriose, xylose, xylobiose, xylotriose and galactose were obtained from the protein database of The Research Collaboratory for Structural Bioinformatics (PDB ID: 2FVY, 3ENG, 1UYY, X, 1B3W, 1UX7 and 2J1A, respectively). The program AutodockTools 1.5.2 ran on Python 2.5 (http://autodock.scripps.edu/) was used to prepare the oligosaccharides and the enzyme for docking. All hydrogen atoms were added and the non-polar hydrogens were merged for all ligands and protein. A number of degrees of torsions of each oligosaccharide were set up to evaluate different thermodynamic properties. A Lamarckian genetic algorithm (16) with different number of energy evaluations and a population size of 150 individuals were applied for docking. The program, Autogrid 4, which pre-calculates grip maps of interaction energies, was used to prepare the grid files, and then docking simulation was performed by Autodock 4 (http://autodock.scripps.edu/). After docking, free energies of binding ΔGb and dissociation constants Kd were reported.

Temperature Stability and pH Optimum.

Temperature stability was measured by incubating 0.2 μg of enzyme for 1 h at nine different temperatures ranging from 25 to 60° C. (Table 9). While GOOX-VN and the variant GOOX-V were stable at 45° C., both lost more than 70% activity after incubation for 1 h at 50° C. The residual activity was measured continuously for 15 min at 37° C. and pH 8 (50 mM Tris-HCl) using 1 mM cellobiose as the substrate, and 0.1 mM 4-aminoantipyrine, 1 mM phenol and 0.5 U horseradish peroxidase to form the chromogenic product with absorbance at 500 nm. The pH stability of GOOX-VN was determined by incubating 0.2 μg of the enzyme for 1 h at pH values from pH 3 to 12. After 1 h of incubation, GOOX-VN retained more than 80% activity at pH 5 to pH 10, 40% activity at pH 4, and less than 10% activity at pH values below 3 or above 11. Finally, the optimum pH for GOOX-VN activity was determined by incubating 0.1 μg of enzyme at 37° C. for up to 5 min with 25 mM cellobiose in 25 mM Britton-Robinson universal buffer solutions at pH 5 to 12. At regular time points, the chromogenic assay mix containing 400 mM potassium phosphate buffer pH 6, 0.1 mM 4-aminoantipyrine, 1 mM phenol, 3 U/ml horseradish peroxidase and 40 mM cellobiose was added to the reaction and was incubated for approximately 5 min at 37° C., until the chromogenic compound was detected. This analysis revealed that the pH optimum of GOOX-VN is pH 10, similar the optimal pH of GOOX-T1 (5).

Site-Directed Mutagenesis.

An additional eight mutations, identified as Y72F, Y72A, E247A, E314A, W351A, N388S, Q353N, Q384A, were created in GOOX-VN to assess the influence of these residues on the substrate selectivity of GOOX-VN. The specific amino acid substitutions were chosen based on substrate docking studies using model structures of the enzyme and substrate. The mutations were generated as previously described; briefly, PCR with the mutagenic primers was performed for 14 cycles of 95° C. for 30 s; 55° C. for 1 min; and 68° C. for 5 min (Table 10). Each variant was recombinantly expressed in P. pastoris as previously described, and purified to more than 95% homogeneity by affinity chromatography. The specific activity of each mutant was then measured at 37° C. for 15 min using 10 mM mono-sugars and 1 mM oligosaccharides as performed for Y300A, Y300N, and W351F (Table 11). This analysis suggests that substituting the Y300 residue may be sufficient to increase the activity of GOOX-VN on different monomeric sugars and linear oligosaccharides, and that E314, Q353 and 0384 are important to the activity of this enzyme. In several cases, mutations led to loss of activity on glucose and xylose, with retention of activity on corresponding oligomeric substrates. This result suggests that corresponding mutations affect the binding and positioning of sugars rather than catalytic mechanism of GOOX-VN.

Chemical Derivatization of GOOX-VN Treated Cellobiose.

To assess the potential of GOOX-VN to direct subsequent chemical derivatization of cellulosic and hemicellulosic substrates, GOOX-VN was used to oxidize cellobiose to its acidic form, and then the carboxyl group of oxidized cellobiose was activated by a carbodimide (N-(3-Dimethylaminopropyl)-N′-ethylcarbodiimide hydrochloride (EDAC)) before it was derivatized by sulfanilic acid (SA) (24). The oxidation of 200 mM cellobiose by GOOX-VN (0.75 μM) was performed in 50 mM Tris-HCl (pH 8.0) at 37° C. for 24 h to maximize the amount of the oxidized product. The enzyme was then removed from the reaction by centrifugation using Nanosep 10K centrifuge filter tubes, and an equal amount of EDAC and SA (83 mM) was added to the remaining reaction components. The consequent chemical derivatization proceeded in the dark for 2 h at room temperature before final reaction products were analyzed by mass spectrometry.

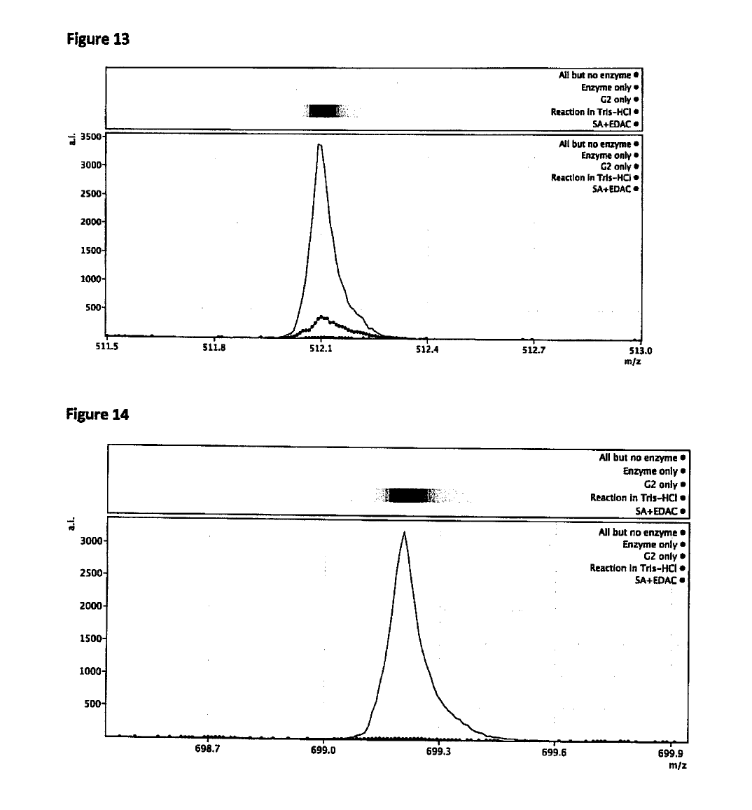

The expected molecular weight of the derivatized product is 512 Daltons, and the generation of the derivatized product only after GOOX-VN treatment was confirmed by mass spectrometry (FIG. 13). It is noted that the activated carboxyl group could also be coupled with other compounds containing other amino groups, including peptide or proteins. Further, in addition to detecting the expected product, a new product with mass to charge ratio (m/z) of 699 was identified in derivatization reactions containing GOOX-VN (FIG. 14). Based solely on its mass, it is anticipated that this product is a dimer of oxidized cellobiose, suggesting that GOOX-VN could be developed to increase the degree of polymerization of cellulosic and hemicellulosic compounds, as well as synthesize novel oligosaccharides and/or polysaccharides.

Nucleotide Sequence Accession Number.

The cloned gene encoding GOOX from Acremonium strictum type strain CBS 346.70 (GOOX-CBS) has been deposited in the GenBank database under accession number GU369974 (http://www.ncbi.nlm.nih.gov/nuccore/GU369974 (Submitted Jun. 15 2010)).

The foregoing description illustrates only certain preferred embodiments of the invention. The invention is not limited to the foregoing examples. That is, persons skilled in the art will appreciate and understand that modifications and variations are, or will be, possible to utilize and carry out the teachings of the invention described herein. Accordingly, all suitable modifications, variations and equivalents may be resorted to, and such modifications, variations and equivalents are intended to fall within the scope of the invention as described and within the scope of the claims.

### REFERENCES

1. Arnold, K., L Bordoli, J. Kopp, and T. Schwede. 2006. The SWISS-MODEL workspace: a web-based environment for protein structure homology modelling. Bioinformatics 22:195-201.
2. Blanchard, V., R. Gadkarl, G. Gerwig, B. Leeflang, R. Dighe, and J. Kamerling. 2007. Characterization of the N-linked oligosaccharides from human chorionic gonadotropin expressed in the methylotrophic yeast Pichia pastoris. Glycoconj J 24:33-47.
3. Boraston, A. B., L. E. Sandercock, R. A. J. Warren, and D. G. Kilburn. 2003. O-glycosylation of a recombinant carbohydrate-binding module mutant secreted by Pichia pastoris. J Mol Microbiol Biotechnol 5:29-36.
4. Cereghino, J. L., and J. M. Cregg. 2000. Heterologous protein expression in the methylotrophic yeast Pichia pastoris. FEMS Microbial Rev 24:45-66.
5. Fan, Z., G. B. Oguntimein, and P. J. Reilly. 2000. Characterization of kinetics and thermostability of Acremonium strictum glucooligosaccharide oxidase. Biotechnol Bioeng 68:231-7.
6. Firbank, S. J., M. S. Rogers, C. M. Wilmot, D. M. Dooley, M. A. Halcrow, P. F. Knowles, M. J. McPherson, and S. E. Phillips. 2001. Crystal structure of the precursor of galactose oxidase: an unusual self-processing enzyme. Proc Natl Acad Sci USA 98:12932-7.
7. Hallberg, B. M., G. Henriksson, G. Pettersson, A. Vasella, and C. Divne. 2003. Mechanism of the reductive half-reaction in cellobiose dehydrogenase. J Biol Chem 278:7160-6.
8. Heuts, D. P. H. M., D. B. Janssen, and M. W. Fraalje. 2007. Changing the substrate specificity of a chitooligosaccharide oxidase from Fusarium graminearum by model-inspired site-directed mutagenesis. FEBS Lett 581:49054909.
9. Hirose, M., S. Kameyama, and H. Ohi. 2002. Characterization of N-linked oligosaccharides attached to recombinant human antithrombin expressed in the yeast Pichia pastoris. Yeast 19:1191-1202.
10. Huang, C. H., W. L Lai, M. H. Lee, C. J. Chen, A. Vasella, Y. C. Tsai, and S. H. Liaw. 2005. Crystal structure of glucooligosaccharide oxidase from Acremonium strictum: a novel flavinylation of 6-S-cysteinyl, 8alpha-N1-histidyl FAD. J Biol Chem 280:38831-8.
11. Huang, C. H., A. Winkler, C. L Chen, W. L Lai, Y. C. Tsai, P. Macheroux, and S. H. Liaw. 2008. Functional roles of the 6-S-cysteinyl, 8alpha-N1-histidyl FAD in glucooligosaccharide oxidase from Acremonium strictum. J Biol Chem 283:30990-6.
12. Kiryu, T., H. Nakano, T. Kiso, and H. Murakami. 2008. Purification and characterization of a carbohydrate: acceptor oxidoreductase from Paraconiothyrium sp. that produces lactobionic acid efficiently. Biosci Biotechnol Biochem 72:833-41.
13. Lee, M. H., W. L Lai, S. F. Lin, C. S. Hsu, S. H. Liaw, and Y. C. Tsai. 2005. Structural characterization of glucooligosaccharide oxidase from Acremonium strictum. Appl Environ Microbiol 71:8881-7.
14. Letourneur, O., G. Gervasi, S. Gaïa, J. Pagès, B. Watelet, and M. Jolivet. 2001. Characterization of Toxoplasma gondii surface antigen 1 (SAG1) secreted from Pichia pastoris: evidence of hyper O-glycosylation. Biotechnol Appl Biochem 33:35-45.
15. Un, S.-F., T.-Y. Yang, T. Inukai, M. Yamasaki, and Y.-C. Tsai. 1991. Purification and characterization of a novel glucooligosaccharide oxidase from Acremonium strictum T1. Biochim Biophys Acta Protein Struct Mol Enzymol 1118:41-47.
16. Morris, G. M., D. S. Goodsell, R. S. Halliday, R. Huey, W. E. Hart, R. K. Belew, and A. J. Olson. 1998. Automated docking using a Lamarckian genetic algorithm and an empirical binding free energy function. J Comp Chem 19:16391662.
17. Parikka and Tenkanen (2009)
18. Parikka, K., A. S. Leppanen, L. Pitkanen, M. Reunanen, S. Willfor, and M. Tenkanen. 2010. Oxidation of polysaccharides by galactose oxidase. J Agric Food Chem 58:262-71.
19. Poirot, O., E. O'Toole, and C. Notredame. 2003. Tcoffee@igs: A web server for computing, evaluating and combining multiple sequence alignments. Nucleic Acids Res 31:3503-6.
20. van Hellemond, E. W., N. G. Leferink, D. P. Heuts, M. W. Fraaije, and W. J. van Berkel. 2006. Occurrence and biocatalytic potential of carbohydrate oxidases. Adv Appl Microbiol 60:17-54.
21. Whittaker (2005)
22. Wohlfahrt et al. (1999)
23. Xu, F., E. J. Golightly, C. C. Fuglsang, P. Schneider, K. R. Duke, L. Lam, S. Christensen, K. M. Brown, C. T. Jorgensen, and S. H. Brown. 2001. A novel carbohydrate:acceptor oxidoreductase from Microdochium nivale. Eur J Biochem 268:1136-42.
24. Mechref, Y., and El Rassi, Z. 1994. Capillary zone electrophoresis of derivatized acidic monosaccharides. Electrophoresis 15:627-634.

TABLE 1
Catalytic activity of GOOX-VN

| | | | | Specific activity^{a} | |
|---|---|---|---|---|---|
| | | | | (μmol mg⁻¹min⁻¹) | |
| | k_{cat} | Kₘ | k_{cat}/Kₘ | | Defined substrate |
| Substrate | (min⁻¹) | (mM) | (mM⁻¹min⁻¹) | From Vₘₐₓ | concentration^{b} |
| Glucose | 449.1 ± 5.5 | 17.36 ± 0.70 | 25.9 ± 1.1 | 7.41 | 2.62 |
| Xylose | 314.9 ± 9.0 | 104.90 ± 10.24 | 3.0 ± 0.3 | 5.20 | 0.42 |
| Galactose | 429.1 ± 8.2 | 131.90 ± 6.58 | 3.3 ± 0.2 | 7.08 | 0.52 |
| NAG | 484.7 ± 12.2 | 340 ± 16.92 | 1.4 ± 0.1 | 8.00 | 0.25 |
| Mannose | ND | ND | ND | ND | ND |
| Maltose | 360.0 ± 5.1 | 2.81 ± 0.16 | 128.3 ± 7.5 | 5.94 | 1.53 |
| Cellobiose | 374.8 ± 10.7 | 0.07 ± 0.01 | 5732 ± 835 | 6.18 | 5.38 |
| Cellotriose | 361.3 ± 11.5 | 0.09 ± 0.01 | 4234 ± 489 | 5.96 | 5.12 |
| Xylobiose | 528.8 ± 5.1 | 0.10 ± 0.00 | 5388 ± 52 | 8.73 | 7.92 |
| Xylotriose | 498.5 ± 7.4 | 0.10 ± 0.01 | 5059 ± 512 | 8.23 | 7.48 |

TABLE 2
Catalytic activity of Y300A

| | | | | Specific activity^{a} | |
|---|---|---|---|---|---|
| | | | | (μmol mg⁻¹min⁻¹) | |
| | k_{cat} | Kₘ | k_{cat}/Kₘ | | Defined substrate |
| Substrate | (min⁻¹) | (mM) | (mM⁻¹min⁻¹) | From Vₘₐₓ | concentration^{b} |
| Glucose | 793.1 ± 14.4 | 8.11 ± 0.40 | 97.8 ± 5.1 | 13.09 | 7.15 |
| Xylose | 680.0 ± 8.2 | 51.84 ± 1.90 | 13.1 ± 0.5 | 11.22 | 1.57 |
| Galactose | 798.1 ± 14.7 | 96.39 ± 5.15 | 8.3 ± 0.5 | 13.17 | 1.15 |
| NAG | 767.8 ± 13.4 | 92.4 ± 4.65 | 8.3 ± 0.4 | 12.67 | 1.1 |
| Mannose | ND | ND | ND | ND | 0.11 |
| Maltose | 624.8 ± 7.4 | 11.00 ± 0.53 | 56.8 ± 2.8 | 10.31 | 0.85 |
| Cellobiose | 823.3 ± 15.5 | 0.24 ± 0.02 | 3366 ± 288 | 13.58 | 10.72 |
| Cellotriose | 666.9 ± 9.5 | 0.25 ± 0.02 | 2667 ± 217 | 11.00 | 9.03 |
| Xylobiose | 797.3 ± 7.3 | 5.11 ± 0.15 | 156.2 ± 4.8 | 13.16 | 2.10 |
| Xylotriose | 832.4 ± 6.6 | 3.15 ± 0.11 | 264.6 ± 9.5 | 13.73 | 3.22 |

TABLE 3
Catalytic activity of Y300N

| | | | | Specific activity^{a} | |
|---|---|---|---|---|---|
| | | | | (μmol mg⁻¹min⁻¹) | |
| | k_{cat} | Kₘ | k_{cat}/Kₘ | | Defined substrate |
| Substrate | (min⁻¹) | (mM) | (mM⁻¹min⁻¹) | From Vₘₐₓ | concentration^{b} |
| Glucose | 648.7 ± 6.2 | 3.11 ± 0.12 | 208.9 ± 8.3 | 10.70 | 8.32 |
| Xylose | 595.9 ± 10.3 | 32.01 ± 1.67 | 21.7 ± 1.2 | 11.48 | 2.32 |
| Galactose | 705.4 ± 5.3 | 109.80 ± 2.29 | 6.4 ± 0.1 | 11.64 | 0.95 |
| NAG | 680.2 ± 6.8 | 55.95 ± 1.93 | 12.2 ± 0.4 | 11.22 | 1.5 |
| Mannose | ND | ND | ND | ND | 0.19 |
| Maltose | 624.0 ± 6.3 | 19.61 ± 0.66 | 31.8 ± 1.1 | 10.30 | 0.49 |
| Cellobiose | 684.2 ± 11.2 | 0.38 ± 0.02 | 1783 ± 98 | 11.29 | 8.15 |
| Cellotriose | 599.0 ± 4.9 | 0.44 ± 0.01 | 1362 ± 33 | 9.88 | 6.90 |
| Xylobiose | 717.5 ± 7.5 | 4.83 ± 0.17 | 148.6 ± 5.5 | 11.84 | 1.83 |
| Xylotriose | 718.2 ± 11.1 | 4.32 ± 0.25 | 165.4 ± 10 | 11.85 | 2.00 |

TABLE 4
Catalytic activity of W351F

| | | | | Specific activity^{a} | |
|---|---|---|---|---|---|
| | | | | (μmol mg⁻¹min⁻¹) | |
| | k_{cat} | Kₘ | k_{cat}/Kₘ | | Defined substrate |
| Substrate | (min⁻¹) | (mM) | (mM⁻¹min⁻¹) | From Vₘₐₓ | concentration^{b} |
| Glucose | 337.3 ± 4.8 | 31.05 ± 1.33 | 10.9 ± 0.5 | 5.57 | 1.31 |
| Xylose | 277.1 ± 3.8 | 288.20 ± 10.14 | 1.0 ± 0.0 | 4.57 | 0.15 |
| Galactose | 393.6 ± 4.5 | 36.12 ± 1.30 | 10.9 ± 0.4 | 6.49 | 1.30 |
| NAG | 467.1 ± 43.1 | 949.5 ± 124.4 | 0.5 ± 0.1 | 7.71 | 0.1 |
| Mannose | ND | ND | ND | ND | ND |
| Maltose | 322.9 ± 4.4 | 4.96 ± 0.23 | 65.1 ± 3.1 | 5.33 | 0.84 |
| Cellobiose | 344.5 ± 5.0 | 0.08 ± 0.00 | 4140 ± 60 | 5.68 | 5.10 |
| Cellotriose | 315.2 ± 6.4 | 0.11 ± 0.01 | 2840 ± 265 | 5.20 | 4.62 |
| Xylobiose | 477.6 ± 3.5 | 0.35 ± 0.01 | 1383 ± 41 | 7.88 | 5.93 |
| Xylotriose | 473.1 ± 4.3 | 0.31 ± 0.01 | 1542 ± 52 | 7.81 | 5.95 |

TABLE 5
PRIOR ART - Oligonucleotide primers
used for gene amplification and
site-directed mutagenesis

| Primer | Sequence |
|---|---|
| EX1* | GCTTCATGGATCCAGGAATTCAACTCAATCAACGCCTG |
| EX2* | TTCAAGTCTAAATCATCTAGATAGGCAATGGGCTCAAC |
| Y300A-F | CAACACCTACTTGGCCGGTGCTGACC |
| Y300A-R | GGTCAGCACCGGCCAAGTAGGTGTTG |
| Y300N-F | CAACACCTACTTGAACGGTGCTGACC |
| Y300N-R | GGTCAGCACCGTTCAAGTAGGTGTTG |
| W351F-F | GCGGCTGGTTCATCCAATGGGACTTC |
| W351F-R | GAAGTCCCATTGGATGAACCAGCCGC |

*From Lee et al. (2005) for gene amplification; others for site-directed mutagenesis.

TABLE 6
Amino acid substitutions in GOOX-VN in comparison
with the GOOX-T1 reported by Lin et al. (1991)

| | Amino acid | GOOX- | | On protein | Distance to sugar |
|---|---|---|---|---|---|
| No. | position | VN | GOOX-T1 | surface* | O¹ (Å) |
| 1 | 23 | E23 | D | Yes | 28.0 |
| 2 | 38 | V38 | A | No | 29.7 |
| 3 | 99 | D99 | N | Yes | 33.7 |
| 4 | 126 | T126 | S | No | 14.3 |
| 5 | 135 | I135 | V | No | 15.0 |
| 6 | 159 | V159 | I | Yes | 24.4 |
| 7 | 175 | K175 | E | Yes | 25.1 |
| 8 | 235 | E235 | Q | No | 22.2 |
| 9 | 259 | Y259 | F | No | 18.3 |
| 10 | 269 | V269 | I | No | 23.2 |
| 11 | 332 | S332 | Q | No | 26.7 |
| 12 | 366 | S366 | A | Yes | 21.2 |
| 13 | 367 | H367 | V | Yes | 20.5 |
| 14 | 388 | N388 | S | No | 11.3 |
| 15 | 435 | D435 | T | Yes | 24.1 |

*Determined by 20% accessible surface area

TABLE 7
The effect of deglycosylation with PNGaseF on enzyme activity

Activity (nmol min⁻¹)*

| Enzyme | Glycosylated | Deglycosylated |
|---|---|---|
| GOOX-VN | 3.51 | 3.43 |
| W351F | 3.13 | 3.19 |
| Y300A | 4.09 | 3.9 |
| Y300N | 2.6 | 2.78 |

*Enzyme activity was measured in duplicate with 10 mM maltose following 15 min at 37° C.

TABLE 8
Free energies of binding (ΔG_{b}) and dissociation constants (K_{d}) of oligosaccharides docked to GOOX-VN enzymes.

| | GOOX-VN | | | Y300A | | | Y300N | | |
|---|---|---|---|---|---|---|---|---|---|
| | Docked | | Distance | Docked | | Distance | Docked | | Distance |
| | energy ΔG_{b} | | to Y429 O^{η} | energy ΔG_{b} | | to Y429 O^{η} | energy ΔG_{b} | | to Y429 O^{η} |
| | (kcal/mol) | K_{d} (μM) | (Å)* | (kcal/mol) | K_{d} (μM) | (Å) | (kcal/mol) | K_{d} (μM) | (Å) |
| Glucose | −5.22 | 149.32 | 2.8 | −5.03 | 204.90 | 2.9 | −5.07 | 191.23 | 2.8 |
| Cellobiose | −6.79 | 10.54 | 2.9 | −6.08 | 34.72 | 2.7 | −6.41 | 20.18 | 2.9 |
| Cellotriose | −6.82 | 10.01 | 2.8 | −6.62 | 14.08 | 2.8 | −6.70 | 12.36 | 2.7 |
| Xylose | −4.70 | 360.24 | 3.1 | −4.60 | 426.36 | 2.9 | −4.62 | 413.78 | 3.2 |
| Xylobiose | −6.24 | 26.60 | 2.6 | −6.01 | 39.22 | 2.8 | −5.93 | 44.68 | 3.2 |
| Xylotriose | −7.49 | 3.24 | 3.0 | −5.73 | 63.11 | 2.9 | −5.87 | 49.52 | 3.2 |
| Galactose | −5.07 | 190.96 | 2.6 | −4.90 | 254.49 | 2.8 | −4.96 | 230.16 | 2.9 |

*Distance between the O^{η} atom of Y429 and the O¹ atom of oligosaccharides. This distance is 2.8 Å in the crystal structure of GOOX-T1 and an inhibitor (PDB ID: 2AXR

TABLE 9
Temperature stability of GOOX-VN

| | Residual activity after 1 h (%)^{a} | |
|---|---|---|
| Temperature (° C.) | GOOX-VN | GOOX-V (N388S substitution) |
| 25 | 100 | 90 |
| 30 | 95 | 100 |
| 35 | 100 | 100 |
| 40 | 100 | 93 |
| 45 | 64 | 86 |
| 50 | 7 | 32 |
| 55 | <5 | <5 |
| 60 | <5 | <5 |

^{a}Average of data from three independent reactions.

TABLE 10
Primers used to generate point mutations

| Mutation | Forward primer | Reverse Primer |
|---|---|---|
| Y72F | GGGTGGTGGTCACAG | CCCATAAGAACCAAA |
| | TTTTGGTTCTTATGG | ACTGTGACCACCACC |
| | G | C |
| Y72A | GGGTGGTGGTCACAG | CCCATAAGAACCAGC |
| | TGCTGGTTCTTATGG | ACTGTGACCACCACC |
| | G | C |
| E247A | CATGCGTCTTGCGAT | GCATTGGCGTTGATC |
| | CAACGCCAATGC | GCAAGACGCATG |
| E314A | CAACTACGACGTCCA | GTTGGCGTAGAAGTA |
| | CGCTTACTTCTACGC | AGCGTGGACGTCGTA |
| | CAAC | GTTG |
| W351A | GCGGCTGGGCTATCC | GTGGAAGTCCCATTG |
| | AATGGGACTTCCAC | GATAGCCCAGCCGC |
| N3885 | GGCAGTTCTACGACA | CGTAGTCGTAGATGC |
| | GCATCTACGACTACG | TGTCGTAGAACTGCC |
| Q353N | CGGCTGGTGGATCAA | GGAAGTCCCAATTGA |
| | TTGGGACTTCC | TCCACCAGCCG |
| Q384A | GCTCTGGCTCTGGGC | CGTAGATGTTGTCGT |
| | TTTCTACGACAACAT | AGAAAGCCCAGAGCC |
| | CTACG | AGAGC |

TABLE 11
Specific activity of additional GOOX-VN mutants on mono-sugars and
oligosaccharides

| | Specific Activity of Enzyme Variants (μmol/min/mg) | | | | | | | | |
|---|---|---|---|---|---|---|---|---|---|
| | GOOX- | | | | | | | | |
| Substrate | VN | Y72F | Y72A | E247A | E314A | W351A | N388S | Q353N | Q384A |
| Glucose | 2.6 | ND^{a} | ND | 0.4 | ND | ND | 1.2 | ND | ND |
| Xylose | 0.4 | ND | ND | ND | ND | ND | 0.3 | ND | ND |
| Galactose | 0.5 | ND | ND | ND | ND | 0.3 | 0.2 | ND | ND |
| NAG | 0.2 | ND | ND | ND | ND | ND | ND | ND | ND |
| Mannose | ND | ND | ND | ND | ND | ND | ND | ND | ND |
| Maltose | 1.5 | ND | ND | ND | ND | ND | 0.4 | ND | ND |
| Cellobiose | 5.4 | 0.2 | 1.1 | 2.2 | ND | 0.6 | 2.4 | ND | 0.2 |
| Cellotriose | 5.1 | 0.5 | 2.1 | 2.7 | ND | 1.1 | 2.1 | ND | 0.4 |
| Xylobiose | 7.9 | ND | 0.5 | 4.2 | ND | 0.1 | 4.3 | ND | ND |
| Xylotriose | 7.5 | ND | 1.3 | 3.5 | ND | 0.1 | 3.4 | ND | ND |
| Xylotetraose | 10.2 | ND | 2.5 | 5.3 | ND | 0.3 | 4.6 | ND | ND |
| Xylopentaose | 10.8 | 0.4 | 3.2 | 5.2 | ND | 0.3 | 4.8 | ND | ND |
| Xylohexaose | 11.0 | ND | 2.2 | 5.1 | ND | 0.5 | 5.0 | ND | 0.1 |

^{a}ND—not detected.
Reactions contained either 10 mM of monosaccharide or 1 mM of oligosaccharide substrate

1. A purified and isolated nucleic acid sequence, or variant thereof, encoding an enzyme having gluco-oligosaccharide oxidase (GOOX) activity, comprising a nucleotide sequence selected from the group consisting of SEQ ID NO. 1, SEQ ID NO. 3, SEQ ID NO. 4 and SEQ ID NO. 5.

2. The purified and isolated nucleic acid sequence of claim 1, wherein the nucleotide sequence comprises SEQ ID NO. 1.

3. The purified and isolated nucleic acid sequence of claim 1, wherein the nucleotide sequence comprises SEQ ID NO. 4.

4. The purified and isolated nucleic acid sequence of claim 1, wherein the nucleotide sequence comprises SEQ ID NO. 5.

5. The purified and isolated nucleic acid sequence of claim 1, wherein the nucleotide sequence comprises SEQ ID NO. 6

6. The purified and isolated nucleic acid sequence of claim 1, wherein the nucleotide sequence is isolated from a strain of Acremonium striatum.

7. The purified and isolated nucleic acid sequence of claim 6, wherein the strain of Acremonium striatum Is CBS 346-70.

8. An enzyme having gluco-oligosaccharide oxidase (GOOX) activity, or a mutant/variant thereof, comprising an amino acid sequence selected from the group consisting of SEQ ID NO. 2, SEQ ID NO. 6, SEQ ID NO. 7 and SEQ ID NO. 8.

9. The enzyme of claim 8, wherein the amino acid sequence comprises SEQ ID NO. 2, and the enzyme is identified as GOOX-VN.

10. The enzyme of claim 8, wherein the amino acid sequence comprises SEQ ID NO. 6, and the enzyme is identified as Y300A.

11. The enzyme of claim 8, wherein the amino acid sequence comprises SEQ ID NO. 7, and the enzyme is identified as Y300N.

12. The enzyme of claim 8, wherein the amino acid sequence comprises SEQ ID NO. 8, and the enzyme is identified as W351F.

13. The enzyme according to claim 9, wherein the enzyme retains at least 60% activity at from 25° C. to 45° C.

14. The enzyme according to claim 9, wherein the enzyme retains more than 80% activity at from pH 5 to pH 10.

15. The enzyme according to claim 9 having an optimal pH of 10.

16. An enzyme having gluco-oligosaccharide oxidase (GOOX) activity, comprising an amino acid sequence that differs from the amino acid sequence SEQ ID NO. 2 by one amino acid substitution.

17. The enzyme of claim 16, wherein the amino acid substitution is selected from the group consisting of Y72F, Y72A, E247A, E314A, W351A, N388S, Q353N, and Q384A.

18. The enzyme claim 8, capable of being used in the oxidation of a substrate.

19. The enzyme of claim 8, capable of being used in the oxidation of a substrate, wherein said substrate is a monomeric or an oligomeric sugar.

20. The enzyme of claim 8, capable of being used in the oxidation of a substrate, wherein said substrate is a monomeric or an oligomeric sugar, wherein said monomeric and oligomeric sugar is a C⁶ or a C⁵ sugar.

21. The enzyme of claim 8, capable of being used in the oxidation of a substrate, wherein said substrate is a monomeric sugar, wherein said monomeric sugar is a C⁶ or a C⁵ sugar, wherein said monomeric sugar is selected from the group consisting of glucose, xylose, galactose, arabinose, mannose and maltose.

22. The enzyme of claim 8, capable of being used in the oxidation of a substrate, wherein said substrate is an oligimeric sugar, wherein said oligomeric sugar is selected from the group consisting of N-acetylglucosamine (NAG), xylo-oligosaccharide and cello-oligosaccharide.

23. The enzyme of claim 8, capable of being used in the oxidation of a substrate, wherein said oxidation enables regio-selective modification of a sugar and/or an oligosaccharide.

24. The enzyme of claim 8, capable of being used in chemical derivatization of cellulosic and a hemicellulosic substrate, or other sugar or oligosaccharide.

25. The enzyme of claim 8, capable of being used in chemical derivatization of cellulosic and a hemicellulosic substrate, or other sugar, wherein said sugar is selected from the group consisting of glucose, xylose, galactose, arabinose, mannose and maltose.

26. The enzyme of claim 8, capable of being used in chemical derivatization of cellulosic and a hemicellulosic substrate, or other sugar, to increase polymerization of said cellulosic and/or said hemicellulosic substrate.

27. The enzyme of claim 8, capable of being used in chemical derivatization of cellulosic and a hemicellulosic substrate, or other sugar, to synthesize a novel oligosaccharide.
